(19) United States
(12) Patent Application Publication
(10) Pub. No.: US 2009/0216254 A1
(43) Pub. Date: Aug. 27, 2009

(54) Device for Preventing Dilation of an Anatomical Lumen or Body Object

(76) Inventor: Peter S. Harris, Bellevue, WA (US)

Correspondence Address: Peter S. Harris, 604 97th Pl SE, Bellevue, WA, 98004, US

(21) Appl. No.: 12/372,186

(22) Filed: Feb. 17, 2009

Related U.S. Application Data
(60) Provisional application No. 61/031,306, filed on Feb. 25, 2008.

(51) Int. Cl. A61B 17/08; A61M 29/00

(52) U.S. Cl. 606/153; 606/191

(57) ABSTRACT

A novel device is provided for the treatment or prevention of a disorder of a patient characterized by regaining lost weight after RNY surgery as a result of dilation failure of the gastro-jujunal stoma. One embodiment the device involves inserting an envelope-like device into the patient's body during or post RNY surgery. Once inserted, the surgeon positions the device to substantially surround the RNY pouch. The device automatically applies a localized compressive force against the proximal Roux limb (jujunum), proportional to the degree of over—fullness of the Roux pouch. Thus a sphincter-like replacement for the pyloric valve is created that substantially reduces the efferent flow of chyme through the g-j stoma, thereby substantially preventing traumatic dilating pressure forces from being imposed on the g-j stoma. The present invention thus uniquely provides a safe and reliable means to ensure the long-term success of the RNY procedure.

### BACKGROUND OF THE INVENTION

1. Field of the Invention

The present invention relates generally to surgical methods and devices, and more particularly to minimally invasive surgical methods and devices for treating obesity in a patient.

2. Background and Description of the Prior Art

The surgical treatment of obesity may be divided into two categories: a) treatment by surgical means to reshape, reconfigure or reconstruct portions of the gastrointestinal tract, for example, by using one of a number of well known “bypass” procedures; and b) treatment by surgical implantation of one or more devices, for example, restrictive devices, electrical stimulation devices, and the like. Broadly, the intention in any of these surgical procedures is to cause weight loss in the patient by either limiting the intake of food or the absorption of nutrients from the food that is eaten, and/or inducing a feeling of early satiety so that the patient eats less.

The surgical procedures well known to the bariatric surgery community today can be categorized into one of three general types: restrictive procedures, where the weight loss is achieved by limiting the amount of chyme accommodated in the stomach; malabsorptive procedures, where the weight loss is achieved by limiting the uptake of nutrients from the ingested food; and combination procedures, involving restriction and malabsorption mechanisms to various degrees.

One of the most common procedures performed, the “Roux -en-Y” gastric bypass (RNY), is of the third type. In the RNY procedure, (created and first performed over 100 years ago by Dr. Cesar Roux (1857-1934), Mont-la-Ville, Switzerland) a small pouch (the restrictive component) is created by cutting and stapling to reconfigure the stomach. To provide the malabsorptive component and re-establish gastro-jujunal continuity, the jejunum is sectioned and re-routed, one end being anastomosed to the pouch in a process more fully described below. The small gastro-jujunal stoma (g-j stoma) so formed functions to regulate the rate at which chyme moves from the pouch into the jejunum. Because of its small size, the pouch makes an abbreviated contribution to the digestive process, acting primarily as a holding vessel for ingested food. Upon completion of the RNY procedure therefore, the patient no longer has a functional stomach; the defunctionalized stomach remnant is disconnected from the path of chyme, although it still produces secretions (ghrelin, etc.) to aid in digestion. In appreciating the mechanism of dilation failure of the RNY, it is important to understand that the operatively created routing of chyme according to the RNY procedure does not provide for a replacement of the sphincter-like function of the pyloric valve which is part of the defunctionalized stomach remnant. Therefore in the RNY system, the g-j stoma bears the full burden of regulating the flow of chyme, a task to which it is structurally unsuited.

The RNY procedure is now almost universally performed laparoscopically, i.e. as a minimally invasive procedure, and because of the acceptable weight loss achieved, it has become the most commonly practiced bariatric surgery in the U.S. A more detailed description of the RNY procedure is given below with reference to FIG. 1.

Other than the RNY, there are a variety of alternative prior art methods and devices for the surgical treatment of obesity. Representative examples from one group of such prior art are described in U.S. Pat. No. 4,403,604, U.S. Pat. No. 6,572,627 and U.S. Pat. No. 7,167,750. All suggest as a primary surgery, a simplistic approach to obesity reduction comprising a constricting wrap or similar device, surgically placed around the whole stomach to constrict it, thereby preventing the patient from ingesting excessive food.

Representative examples from another group of such prior art include U.S. Pat. No. 6,511,490, U.S. Pat. No. 6,547,801 and U.S. Pat. No. 7,118,526. All suggest as a primary surgery an approach to obesity reduction comprising surgically placing a band (commonly known as a laparoscopic gastric band) so as to form a constriction around the upper part of the whole stomach, distal to the esophagus. Said gastric band creates a small proximal pouch designed to limit the amount of food ingested, and the constriction slows the passage of chyme through the whole stomach.

The success of the RNY procedure in causing weight loss is dependent on its ability to regulate (i.e. reduce the rate of) the flow of chyme from the pouch through the restricting g-j stoma, according to Bernoulli's principles describing fluid flow. Beyond the short-term and long-term complications that may be associated with the RNY surgery, the most common cause of failure of the RNY is dilation of the g-j stoma, which results in a loss of the ability to regulate this flow, leading to a reduction or even reversal of weight loss in the patient. Long-term clinical studies have shown the occurrence of such failures in RNY patients may be significant after only a few years, and this has severe implications, not only for patient's ability to maintain permanent weight loss, but also in terms of the costs and additional risks of complications associated with follow-up care and any corrective surgical interventions that may be required.

Surgically re-forming the RNY system to a smaller volume, or “converting” the RNY to another form of bypass is usually a major “open” surgical procedure infrequently done because of the many surgical risks well known to the surgical community, and very high financial cost to the patient.

Treatment of a dilated RNY system by minimally invasive intragastric means (i.e. operating entirely within the pouch) with access to the operative site being gained via the esophageal route has been attempted. For example U.S. Pat. No. 5,792,153 and U.S. Pat. No. 6,835,200 describe devices that could be used to treat the dilation of the pouch of a failed RNY system via esophageal access. Other commercial devices addressing this problem include the SewRight™ (LSI Solutions, Inc., Victor, N.Y.), and the StomaphyX™ (EndoGastric Solutions, Inc, Redmond, Wash.). In all cases, these devices attempt to form one or more plications within the pouch, thus reducing the pouch volume, securing them by proprietary fastening means.

The aforementioned surgical interventions for treating a dilated RNY system are all disadvantaged by numerous problems and post operative complications, some potentially life threatening, that are well known to practicing bariatric surgeons. Furthermore, none of the above-described treatments can be performed as a prophylactic, at the time of the primary RNY surgery, thus eventually subjecting patients to the operative risk and financial cost of a rescue operation. Consequently, patients with a dilation-failed RNY have had no realistic hope of amelioration of their condition.

Given the current situation described above, there is a need for a safe, easy-to-perform, efficacious and cost-effective surgical procedure, and associated enabling surgical devices, for the minimally invasive treatment or prevention of said condition.

### BRIEF SUMMARY OF THE INVENTION

The present invention provides a completely new solution for the surgical treatment of obesity, more particularly to methods and devices for preventing the failure of RNY systems caused by dilation of the g-j stoma. To appreciate the novelty and non-obviousness of the present invention, it is necessary to understand stomach anatomy, the nature of the dilation failure mechanisms of RNY systems, and the mechanics of fluid flow as applied to the upper gastrointestinal tract, as discussed below.

For descriptive purposes, the stomach is commonly divided into several regions, known to those in the art as the lesser curve, fundus, greater curve, gastro-esophageal junction, and cardia. In the commonly performed version of the RNY procedure, the size of both the g-j stoma and pouch have become somewhat standardized by the surgical tools used to create them. The g-j stoma is typically created in the size of approximately 15 or 20 mm diameter, depending on the size of tool chosen. The pouch is typically created to encompass a volume of about 15-20 ml.

In is widely known that the preoperative whole stomach has the ability to dilate flexibly and reversibly in response to overeating, with some regions able to accommodate stretching more than others. The RNY pouch is always constructed in the area near the gastro-esophageal junction and cardia, regions of the stomach that, unfortunately, are least able to stretch elastically and reversibly in response to overeating. Repeated dilation therefore often causes permanent plastic (i.e. non-recoverable) expansion of the RNY system. Specifically, over time (e.g. several years post operatively) repetitive patient overeating causes the pouch to increase in size to the range of 50-100 ml, and forced passage of chyme under elevated pressure causes the g-j stoma to dilate to about 30 mm diameter. This is a common failure mode of RNY systems, manifesting itself as regaining of weight loss, a condition referred to as recidivism.

Currently, there is no easily performed surgical procedure to re-form the dimensions of the RNY system to its desirable immediate post-operative dimensions, and no known method to prevent said dilation that can be applied as part of the primary procedure. This is a major shortcoming of the RNY procedure, addressed by the present invention.

The flow, F, of food through the g-j stoma depends on two factors, i.e.

F=A×P (i)

where A is cross sectional area and P is the pressure forcing food through the g-j stoma. If D is the diameter of the g-j stoma, then

A=π/4×D² (ii)

Surprisingly, the flow F does not depend on the volume of the pouch. Thus, attempting to repair a dilated RNY system by reducing pouch volume alone (the primary goal of many prior art methods and devices) only forces more food thru the stoma (due to increased P), and if the stoma were also reduced as part of the repair, dilates it again.

It has therefore unexpectedly been found, according to the present teachings, that in order to maintain the long-term efficacy of the stoma in regulating flow in RNY systems, it is necessary to protect the diameter of the g-j stoma using mechanical restraint.

In a novel inventive step, the present invention provides such restraint by exerting sphincter-like control of the efferent flow of chyme, thus substantially reducing the pressure P exerted on the g-j stoma.

To illustrate how the diameter of the g-j stoma exerts an unexpected amplified effect on the flow of chyme, consider the following example. Increasing the g-j stoma diameter from 20 mm post operatively to 30 mm at failure is not simply a 50% increase in flow F, but because cross sectional area depends on the square of the diameter, it is surprisingly a 125% increase. Therefore the flow of chyme/food into the jujunum is 125% greater in this example of a dilation failure, and is unexpectedly exacerbated by a small pouch.

Therefore, according to the present invention, minimally invasive methods and devices are provided for preventing the dilation of a lumen or other bodily organ. Of particular interest with respect to the treatment of obesity, such methods and devices are used in conjunction with RNY systems that have been dilated over time (i.e. as a repair operation), or to prevent such dilation from occurring (i.e. applied as a prophylactic during the initial RNY procedure). In both of these applications, the method generally includes positioning an envelope-type structure around the Roux system, including the proximal Roux-limb. The envelope-type structure is capable of automatically modulating the amount of chyme passing through the g-j stoma into the Roux-limb, thereby preventing the g-j stoma from dilating undesirably over time.

Preferably, the structure of the present invention is constructed from material soft enough to prevent abrasions but that does not stretch appreciably, thus substantially restricting dilation of the underlying anatomical structure to which it is applied. Although the descriptions that follow describe an envelope type configuration as the most preferred embodiment in the RNY application, it is to be understood that there are many other methods of utilizing the stretching stomach to vary the efferent flow of the stomach that are within the scope of this invention. For example a tape, band or strap or partial envelope or other means suitably constructed according to the principles of the present invention, affixed to the stomach and routed over the proximal Roux limb could accomplish the same, as could a tape, band or strap or partial envelope or other means similarly routed over the g-j stoma, and yet another embodiment could be provided that accommodates a pouch substantially larger than the usual 15-20 ml volume.

In general, it is preferable that the structure be collapsible into a pre-deployed configuration, to facilitate loading into a minimally invasive delivery device. Once inserted into the body, e.g. using laparoscopic or endoscopic methods known to those in the art, the envelope-type structure is expanded to its deployed configuration, and manipulated into position to at least substantially surround a portion of the anatomical structure of interest, whereupon it is secured or otherwise fastened to ensure it remains in place. In the case of RNY systems, the envelope-type structure is deployed to at least substantially surround a portion of the RNY pouch, including at least a section of the Roux limb immediately proximal to its attachment to the pouch (i.e. near the gastro-jujunal anastomosis site).

The present invention directly addresses by primary intent the most significant failure mode of the RNY-dilation of the g-j stoma. It overcomes the limitations and shortcomings of the prior art in several ways. Firstly, it is safe and easy to install in a minimally invasive manner. Secondly, it may be installed prophylactically as part of the primary procedure or during a subsequent repair procedure. Thirdly, when installed as part of the primary procedure it will positively prevent the RNY system from failing under incorrigible overeating, Lastly it can be installed at a minimal cost to the patient and a minimal effort by the surgeon, and can be reversed (removed) in a simple surgical procedure.

Further objects and advantages are to provide a means for a human operator to control or otherwise beneficially supplement the functionality of the system, e.g. by communicating with one or more adjustment or control means located outside the body by using either wired or wireless technology.

While the present invention will be described more fully hereinafter with reference to the accompanying drawings, in which particular embodiments are shown and explained, it is to be understood that persons skilled in the art may modify the embodiments herein described while achieving the same methods, functions and results. Accordingly, the descriptions that follow are to be understood as illustrative and exemplary of specific structures, aspects and features within the broad scope of the present invention and not as limiting of such broad scope.

### BRIEF DESCRIPTION OF THE DRAWINGS

FIG. 1 is a schematic diagram of a postoperative RNY stomach and pouch illustrating the revised routing of chyme through the digestive tract.

FIG. 1A shows an alternative surgical routing of the RNY digestive tract.

FIG. 2 illustrates a view in plan of a first embodiment of the apparatus, after deployment from the collapsed delivery state, now in the flat state ready for the surgeon to wrap around the Roux system.

FIG. 3 shows a plan view of the first embodiment of the apparatus overlaid over a plan view of an RNY pouch and Roux limb.

FIGS. 4, and 5 illustrate how the first embodiment of the apparatus selectively compresses the Roux limb proximal to the gj stoma thus limiting the stretching forces imparted to the g-j stoma.

FIGS. 6 and 7 illustrate how a second embodiment of the enveloping apparatus constricts the Roux limb proximal to the g-j stoma in the case of an alternative surgical approach, thus limiting the stretching forces imparted to the g-j stoma.

### DETAILED DESCRIPTION OF THE INVENTION

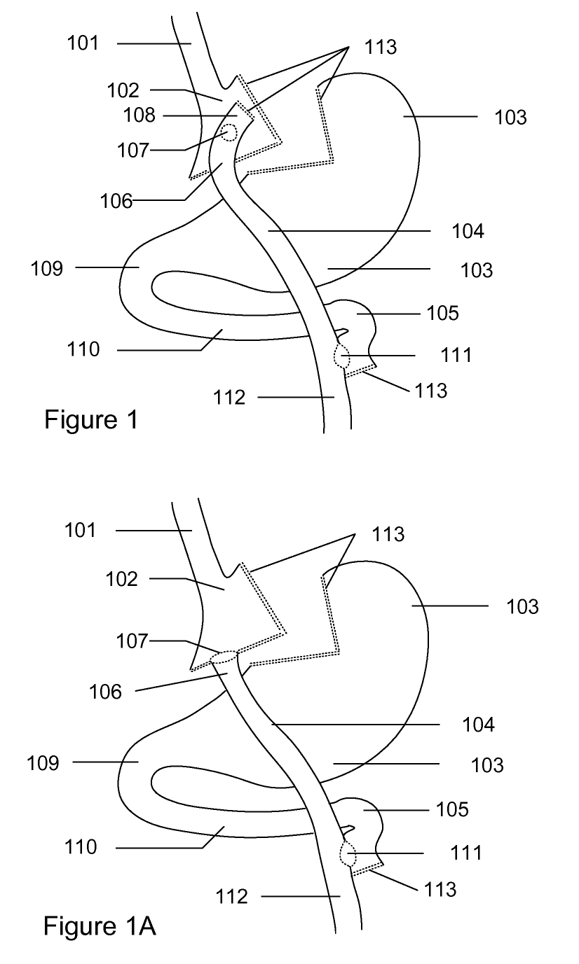

A more detailed outline of the RNY bypass procedure will be helpful in appreciating how the present invention prevents the failure mode previously described noting that in the following description FIGS. 1, 2, 3, 4, and 5 pertain to an embodiment herein described; while FIGS. 1A, 6 and 7 pertain to a second embodiment herein described.

FIG. 1 and FIG. 1A illustrate how the stomach is divided into two sections in two common variants of the RNY surgical procedure. The placement of the g-j stoma 107 differentiates these variants as further described below, and necessitates a corresponding variant in the present invention, also described below.

In the RNY procedure, a small pouch 102 is operatively created and now becomes the functional stomach, receiving chyme from the esophagus 101. The stomach remnant 103, is left in situ but plays no further part in transporting or holding, or processing chyme. Two additional steps complete the operation: firstly the small intestine in its jujunal region, 104, is divided and one end called the “Roux limb” 106, is anastamosed to the pouch 102, with an opening 107 (the g-j stoma) between them to re-establish gastro-intestinal continuity. (Note that in the case of an alternative surgical approach shown in FIG. 1A, the g-j stoma 107 is constructed through the horizontal staple line; other than this variation, routing and flow of chyme remains the same). Now the route of chyme is from the pouch 102 via said stoma directly into the jejunum 104 and no longer via the pylorus 109. The mechanics of the above described procedure in the first surgical variant result in a short blind stub 108 of the jujunum that plays no part in the conduction of chyme. The last operative step is to anastamose the Roux limb to the pancreato-billiary limb thus forming the other arm of the Y, 105. The pancreato-billiary limb 110 allows for the normal drainage of ghrelin, a hormone produced by the stomach, gastric juices, pancreatic juices and bile, continuity being provided to the jejunum via the jujuno-jujunal stoma 111. The leg of the Y, the “common channel” 112, carries the chyme onward to the ileum (not illustrated) and hence the colon (not illustrated) in the normal way. Division and closure of tissue in the RNY procedure can be done by hand or more commonly, with specialized stapling tools. The present invention will work equally well in both cases.

It will be noted that the operatively created path for chyme does not include any sphincter-like mechanism to replace the pyloric valve, a deficiency of the RNY procedure that is addressed by the present invention.

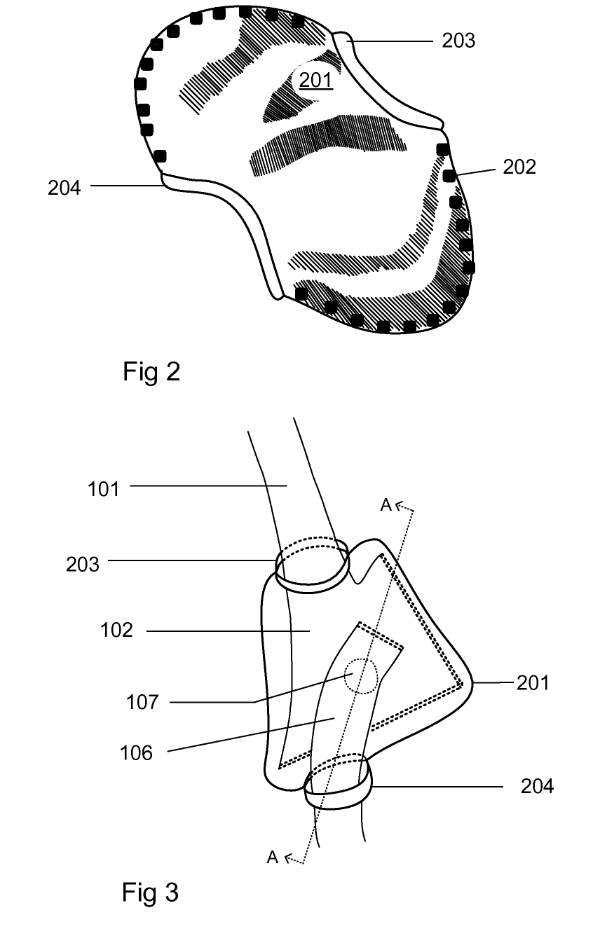

FIG. 2 illustrates the first herein described embodiment of the apparatus in plan view after deployment from the collapsed delivery state. In this deployed flat state it is ready for the surgeon to fold around the Roux system. Preformed contours 201, illustrated by hatching in the illustration, allow the apparatus to conform to the shape of the anterior and posterior and lateral aspects of the pouch respectively.

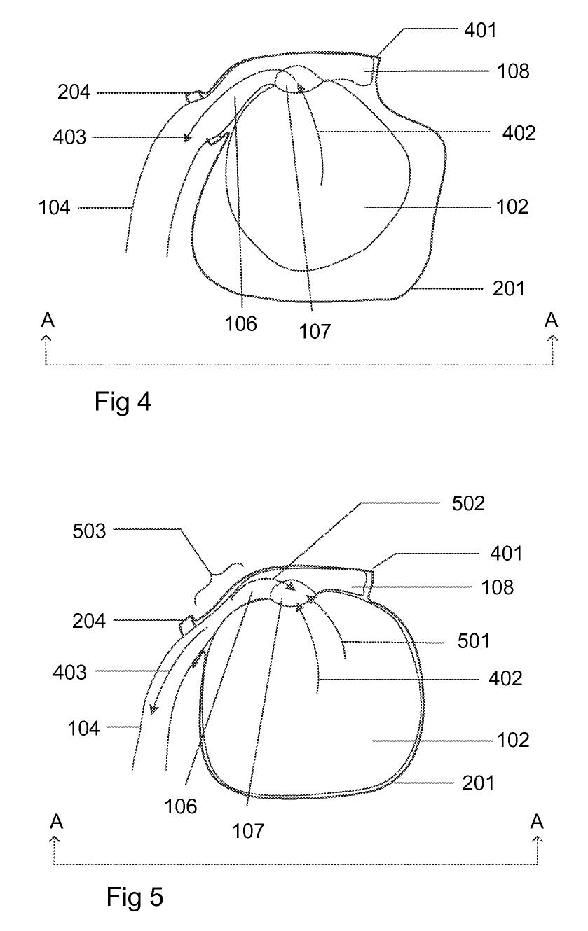

The preformed shape referred to here causes the selective compression of the Roux limb as described more fully below with reference to FIGS. 4 and 5.

FIG. 3 shows a plan view of the same embodiment of the apparatus 201 being overlaid over a plan view of an RNY pouch and Roux limb, showing the esophagus 101, pouch 102, gj stoma 107, and Roux limb 106. In FIG. 3, the surgeon had deployed the apparatus from its collapsed state (necessary for delivery to the operative site via a laparoscopic trocar) to its flat state, and then positioned it so that it envelops the Roux system. The shaped cuff-like edges 203 and 204, when deployed by the surgeon, conform to, and loosely encircle, the esophagus and Roux limb respectively, thus holding the apparatus in situ. Said cuff-like edges provide a large footprint to reduce the likelihood of abrasion and irritation in the area of contact with the patient's tissue. Those skilled in the art will note that there is therefore no need for the device to be sutured, stapled or otherwise fastened to the patient's tissue, thereby avoiding likely injury to tissue from said fastening means. It will be further noted that while in the particular embodiments illustrated here there is no need or provision to secure or fasten the apparatus to the patient's tissue, in other embodiments it might be beneficial and may be provided for by means obvious to those skilled in the art.

Once folded around the Roux system as illustrated in FIG. 3, the apparatus is be held in situ by a means selected from a multiplicity of snaps, hooks, sutures, and the like, representatively illustrated by 202, and a further similar fastening means (not illustrated) fastening the cuff-like edges 203 and 204.

Section AA projected in FIGS. 4 and 5 show section AA when the pouch is less than full, and full to the overeating level, respectively.

Thus, as illustrated in FIG. 4, when the pouch is substantially empty, it causes a minimal pressure of chyme against the proximal entry to the stoma. Therefore, chyme flows at rate and volume mainly limited by the diameter of the gj stoma 107, and is not limited by the cuff-like edge 204, which as describe above fits loosely over the jujunum. Arrows 402 and 403 represent this flow.

In appreciating the functioning of the aforesaid mechanism, it is important to recall that as previously described, a) the enveloping apparatus 201 does not significantly expand or stretch, b) the protruding contour of the Roux limb (i.e., the proximal efferent jujunum), 106 is accommodated by the matching contour 401 in the apparatus, c) said matching accommodation prevents the Roux limb from being compressed enough to prevent all flow of chyme, and d) in the absence of the present invention, the operatively created system does not contain a replacement for the now excluded pyloric valve.

(Note that in FIGS. 4 and 5, 6 and 7, arrows 402 and 403 represent flow; and arrows 501 and 502 represent pressure.

FIG. 5 illustrates the case of overeating and how the present invention provides a functional replacement for the pyloric valve. Since in this case chyme cannot exit the pouch 102 as fast as food is ingested, the pouch dilates until the enveloping apparatus, 201, restrains it. In this condition, the pouch compresses the Roux limb (jujunum) 106, against the relatively immovable enveloping apparatus in the zone 404. The resulting back-up of chyme in zone 404 causes an afferent- or back-pressure 502 that is slightly less in amplitude than the efferent- or emptying pressure 501. Thus the pressure gradient across the g-j stoma is substantially reduced, thereby reducing the force that causes dilation failure in the absence of the current invention.

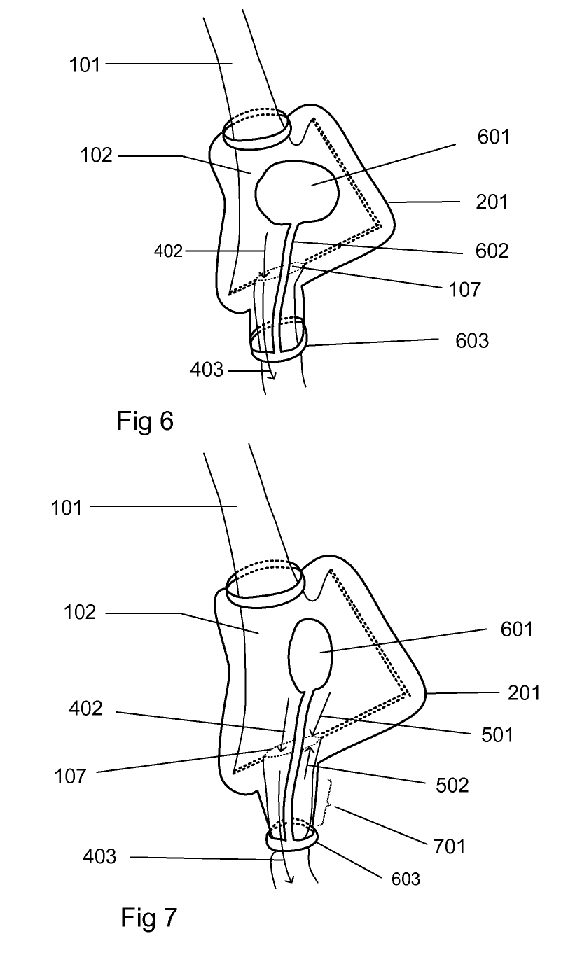

FIG. 6 illustrates, with reference to FIG. 1A, a second embodiment for the case when the surgeon uses this surgical approach. In this embodiment, 601 is a flexible, compressible reservoir containing a fluid such as saline, connected by a flexible tube or conduit 602 to the actuatably restrictive cuff 603 that loosely encircles the jujunum, these elements forming an hydraulic actuator system that automatically modulates the efferent flow of chyme.

Thus, as illustrated in FIG. 6, when the pouch is substantially empty, it causes a minimal pressure of the pouch against the reservoir 601. Therefore, chyme flows at rate and volume mainly limited by the diameter of the g-j stoma 107, and is not limited by the restrictive cuff 603, which as describe above fits loosely over the jujunum. Arrows 402 and 403 represent this flow.

FIG. 7 illustrates the case of overeating and how the second embodiment of the present invention provides a functional replacement for the pyloric valve. Since in this case chyme cannot exit the pouch 102 as fast as food is ingested, the pouch dilates until the enveloping apparatus, 201, restrains it. In this condition, the pouch compresses the reservoir 601 against the relatively immovable enveloping apparatus. Conduit or tube 602 transfers the resulting hydraulic pressure to cuff 603 causing it to exert a constrictive force on the jujunum. The resulting back-up of chyme in zone 701 causes an afferent—or backpressure 502 that is slightly less in amplitude than the efferent—or emptying pressure 501. Thus the pressure gradient across the g-j stoma 107 is substantially reduced, thereby reducing the force that causes dilation failure in the absence of the current invention.

It will be apparent to those skilled in the art that it would be possible or even desirable to augment said hydraulic force with electrically driven or other means, and further, to provide means for the patient or other human operator to beneficially assist or influence said modulation.

In both embodiments described above, and with reference to equation (i): Since the rate of flow (F) is determined by the diameter (D) of the stoma and the efferent pressure (P) on the proximal entry to the stoma, it can be readily understood that P rapidly increases as more food entering the pouch is forced against the stoma. (When this condition occurs in a RNY system not treated by the present invention, it is the direct cause of the undesirable plastic (i.e. non-recoverable) dilation of the stoma referred to previously).

In a novel and inventive step claimed by the present invention, said back-up of chyme causes an afferent or back pressure (P′) represented by arrow 503, almost equal to efferent pressure (P) represented by arrow 505. This can be expressed as:

P-P′=Δ (iii)

where Δ is the resulting small pressure gradient across the stoma. Said small gradient is insufficient to cause dilation of the stoma, moreover, it is desirable since it enables the necessary beneficial continuous outflow of chyme (represented by arrow 506) required to ensure eventual emptying of the pouch.

Reducing the pressure gradient in the novel manner described has the beneficial effect of directly negating the cause of dilation failure of the stoma in the RNY system.

What I claim is:

1. An implantable system comprising a flat body of material that when deployed forms an envelope around an anatomical object, shaped to form at least one port or aperture once deployed, and further comprising: a) at least one means to close, and a′) secure in place said envelope .

2. The system of claim 1 wherein the shape and dimensions of said system are such that it beneficially exerts substantially more or less pressure influence on selected components, perturbations or extremities of the anatomical object than on others.

3. The system of claim 1 wherein said pressure influence is modulated by the amount of chyme, blood or other biological fluid ingested or otherwise carried into said anatomical object.

4. The system of claim 1 comprising in combination an end effector and means for influencing the dimensions or functionality of selected parts of said, or other, lumen or anatomical object.

5. The system of claim 1 comprising in combination an end effector and controlling means for a human to control or otherwise beneficially supplement the functionality of the system.

6. An implantable system comprising a substantially flat body of material wherein selected areas or zones are preshaped, hardened, softened, or braced or the like, such that when deployed it forms an envelope comprising uneven contours about its surface, and shaped to form at least one port or aperture once deployed, and further comprising: a) at least one means to close, and a′) to secure in place said envelope.

7. The system of claim 6 wherein the shape and dimensions of said system are such that it beneficially exerts substantially more or less pressure influence on selected components, perturbations or extremities of the anatomical object than on others.

8. The system of claim 6 wherein said pressure influence is modulated by the amount of chyme, blood or other biological fluid ingested or otherwise carried into said anatomical object.

9. The system of claim 6 comprising in combination an end effector and means for influencing the dimensions or functionality of selected parts of said, or other, lumen or anatomical object.

10. The system of claim 6 comprising in combination an end effector and controlling means for a human to control or otherwise beneficially supplement the functionality of the system.

11. An implantable system comprising a substantially flat body of material wherein selected areas or zones are preshaped, hardened, softened, or braced or the like, such that when deployed it forms an envelope such that while the whole is one envelope, its interior space is divided into a plurality of chambers, cavities, hollows, compartments, spaces, slots and the like, and shaped to form at least one port or aperture once deployed, and further comprising: a) at least one means to close, and a′) secure in place said envelope.

12. The system of claim 13 wherein the shape and dimensions of said system are such that it beneficially exerts substantially more or less pressure influence on selected components, perturbations or extremities of the anatomical object than on others.

13. The system of claim 13 wherein the shape and dimensions of said system are such that it beneficially exerts substantially more or less pressure influence on selected components, perturbations or extremities of the anatomical object than on others.

14. The system of claim 13 wherein said pressure influence is modulated by the amount of chyme, blood or other biological fluid ingested or otherwise carried into said anatomical object.

15. The system of claim 11 comprising in combination an end effector and means for influencing the dimensions or functionality of selected parts of said, or other, lumen or anatomical object.

16. The system of claim 13 comprising in combination an end effector and controlling means for a human to control or otherwise beneficially supplement the functionality of the system.
